(19) United States
(12) Patent Application Publication
(10) Pub. No.: US 2009/0131891 A1
(43) Pub. Date: May 21, 2009

(54) Flexible cover for use in flushing of peripheral organs

(76) Inventor: Peter Fons, Christiansfeld (DK)

Correspondence Address: JAMES C. WRAY, 1493 CHAIN BRIDGE ROAD, SUITE 300, MCLEAN, VA, 22101, US

(21) Appl. No.: 11/921,910

(22) PCT Filed: Jun. 9, 2006
(86) PCT No.: PCT/DK2006/000320

§ 371 Date: Dec. 26, 2007

(30) Foreign Application Priority Data
Jun. 10, 2005 (DK) PA 2005 00833

(51) Int. Cl. A61M 35/00

(52) U.S. Cl. 604/311; 604/310

(57) ABSTRACT

The apparatus (1) according to the invention for flushing a surface (6) with a fluid comprises a fluid supply (2) connected to a hand held unit (4) for supply of pressurised fluid to the hand held unit (4). The hand held unit (4) is configured to provide a jet (14, 14′) of fluid towards the surface (6) and is connected via a connector (15) to a cover (5). The cover (5) is provided with a rim part (9) that is resilient for easy adaptation of the rim (10) to a non-planar surface (6) that is to be flushed. The hand held unit (4) with the cover (5) has means for changing the direction of the jet (14, 14′) in the cover (5) for flushing an area under the cover (5) which largely corresponds to the entire area covered by the cover (5).

### FIELD OF THE INVENTION

The invention relates to a cover in connection with an apparatus for flushing peripheral organs in humans and animals, wherein liquid is conveyed from a reservoir under pressure to a discharge branch via a hose or the like to the peripheral organs. Such a flexible cover is e.g. particularly suitable during cleaning of contaminated wounds caused e.g. by fall accidents.

### BACKGROUND OF THE INVENTION

In European patent application EP 1 372 755 by Føns assigned to Curatio Aps, an apparatus for flushing peripheral organs in humans and animals is diclosed. The apparatus comprises a pulsator between a liquid reservoir and a hand held unit with a nozzle in order to provide pulsating liquid out of the nozzle. As the liquid flow is pulsating, a more effective cleaning is achieved than if a smooth flow were used. The nozzle is surrounded by a protective shield preventing unintentional spreading of liquid which may contain impurities in the form of bacteria.

When using this apparatus over a relatively large area of the skin, the shield is moved along the skin in order to flush the complete area. However, this is experienced as painful by the patient, which is a disadvantage. On the other hand, if the flushing shield is lifted, the region around the wound is not tightly closed and water droplets may leave the shield which is undesired and therefore another disadvantage.

### DESCRIPTION/SUMMARY OF THE INVENTION

It is therefore the object of the invention to provide an improvement to the apparatus as disclosed in EP 1 372 755 to avoid the disadvantages. It is a further purpose of the invention to provide an improved shield without the above mentioned disadvantages. This object is achieved with an apparatus and a cover according to the independent claims.

The apparatus according to the invention for flushing a surface with a fluid comprises a fluid supply in connection with a hand held unit providing a jet of fluid towards the surface and is connected via a connector to a cover. The cover is provided with a rim part that is resilient for easy adaptation of the rim to a non-planar surface that is to be flushed. The hand held unit with the cover has means for changing the direction of the jet in the cover for flushing an area under the cover which largely corresponds to the entire area covered by the cover.

Because of the means for changing the direction of the jet in the cover, the flushing cover needs not be lifted and moved over the region to be flushed. Thus, the cover tightly encloses the region such that no water droplets leave the cover. Furthermore, pain due to movement of the cover along the surface of a wound to be flushed is prevented. Also, in connection with chronic sores, the invention is highly beneficial, because the cover prevents spread of multi-resistant bacteria, a severe problem in many hospitals.

In an embodiment of the invention, the means for changing the direction of the jet in the cover comprises a flange part of the cover with a flexing zone between the rim part and the connector for large angle tilt of the hand held unit with respect to the rim part.

Other possibilities for changing the direction of the jet may be envisaged such as tiltable nozzles in the hand piece or a turnable tube with a fan-like jet.

The connector may have various forms, for example comprise a solid, transparent shield. Such a shield may, for example, be formed as a semi-sphere. Preferably the connector is made of plastic. It may be available as a one-way product connected to a likewise disposable flexible pipe, which in turn is connected to the fluid supplying apparatus and wherein the connector is connected to the cover. After use, the connector and the pipe may be thrown away instead of getting cleaned. The cover may also be made of a disposable one-time-use material. However, as silicone rubber usually is more expensive than the typically used plastic for the connector, re-use after cleaning in an autoclave may be considered.

The connector may comprise a magnifying lens for ease of observation of the flushed surface, for example a wound. It should be notified that a magnifying lens is a great advantage in connection with the invention, but may also with benefit be used in shields of other types.

The invention also concerns a cover for an apparatus as just described, wherein the cover has an opening for receiving a connector, a resilient rim part for adapting the rim to a non-planar surface to be flushed, and a flange part with a flexing zone between the rim part and the connector for large angle tilt of the connector with respect to the rim part. The flexing zone may comprise an area with corrugations, for example encircling the connector in order to obtain free movement in all tilt directions.

Experiments have shown that flexible silicone rubber is a good material, such that at least the rim part of the cover is made thereof, but preferably the entire cover is made thereof.

The cover according to the invention may have varying sizes in dependence of the application. For flushing peripheral organs, for example skin with wounds, it has turned out to be of advantage if the diameter of the rim is between 120 mm and 180 mm, preferably between 130 an 170 mm and most preferably around 170 mm.

The cover according to the invention may be used in an apparatus as disclosed in the aforementioned patent application EP 1 372 755 where the fluid supply is connected to the hand held unit via a tube. Another example is a syringe as the hand held unit, where the syringe itself contains the fluid supply. The connector in this case may comprise a coupled to the syringe

### SHORT DESCRIPTION OF THE DRAWINGS

The invention will be explained in more detail with reference to the drawing, where

FIG. 1 is a schematic of an apparatus according to the invention,

FIG. 2 is a drawing of the cover according to the invention.

FIG. 3 is an illustration for the mounting of such a cover

FIG. 4 illustrates the flexible nature of the cover,

FIG. 5 illustrates alternative embodiments according to the invention.

### DETAILED DESCRIPTION/PREFERRED EMBODIMENT

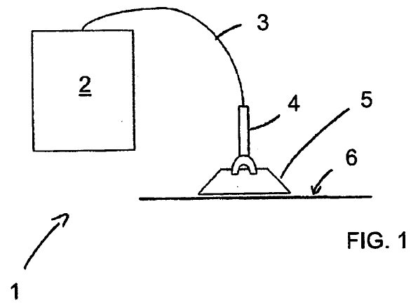

FIG. 1 illustrates an apparatus 1 according to the invention having a liquid supply 2 with a reservoir which via a hose 3 is connected to a hand held unit 4. For example, such an apparatus could be constructed as it is explained in European patent application EP 1 372 755 by Føns assigned to Curatio Aps.

According to the invention, the hand held unit 4 comprises a flexible cover 5 to be placed on a surface 6 to be flushed, for example the skin of a person or of an animal. When liquid is provided to the surface 6, for example a pulsating liquid, the cover 5 prevents droplets to leave the volume between the cover 5 and the surface 6, for example the skin of a person or animal. A typical application is the cleaning of wounds on peripheral organs.

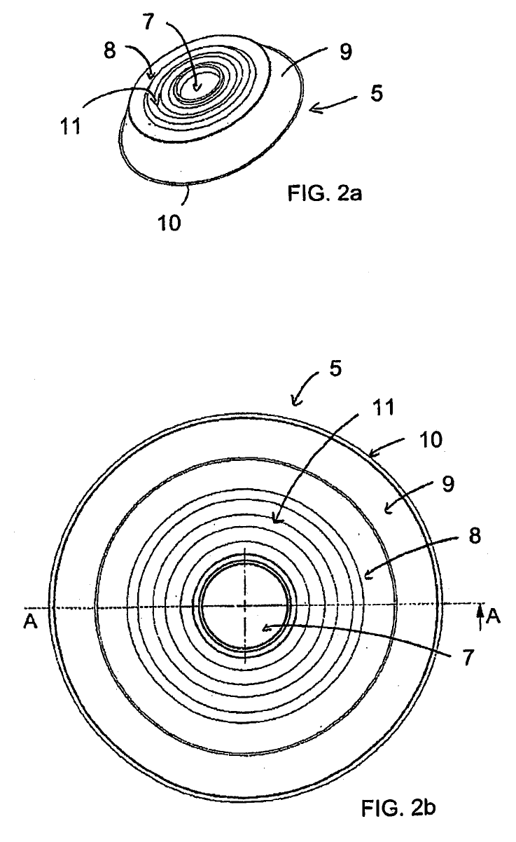

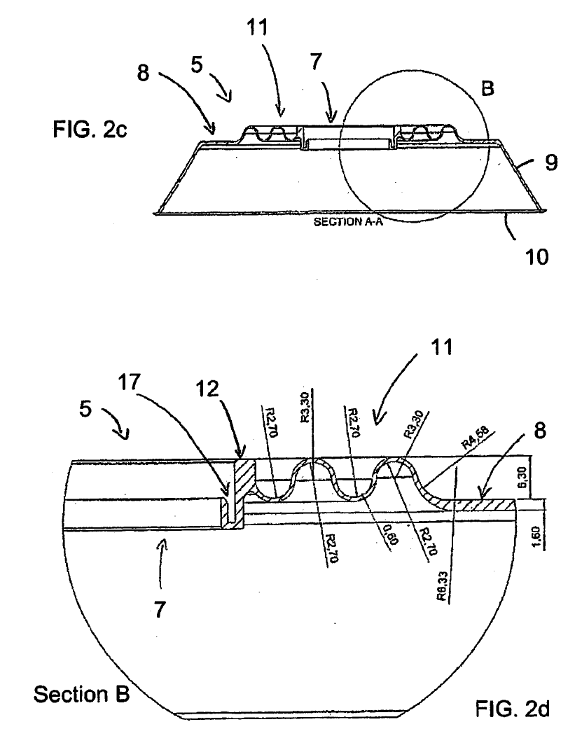

The cover according to the invention is shown in more detail in FIG. 2a-d. FIG. 2a illustrates the cover 5 in a perspective view, FIG. 2b in a top view, FIG. 2c in a cross sectional view along the line A-A as illustrated in FIG. 2b, and FIG. 2d shows the circular cut of FIG. 2c in an enlarged view. The indicated dimensions in FIG. 2d are only exemplary and do not limit the invention in any way.

The cover 5 according to the invention comprises a flange 8 and a conical side wall 9. The flange 8 comprises an opening 7 through which flushing fluid is supplied. The cover 5 is produced in a flexible material such that its rim 10 adopts its shape to the surface 6 on which it is placed, for example a limb of a patient.

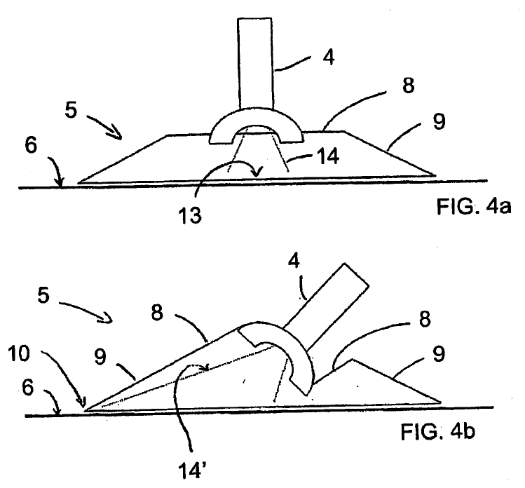

The flange 8 has a corrugated part 11 which allows great mechanical flexibility of the rim of the opening 12 relative to the conical part 9. This is illustrated in FIGS. 4a and 4b. In FIG. 4a, the situation is shown, where a jet 14 of fluid, for example water, leaves the hand held unit 4 and flushes the central part 13 of the surface 6 inside the cover 5. In contrast to a solid shield or a semisolid shield without a high flexibility, it is possible with a cover according to the invention to tilt the hand piece 4 with respect to the conical side wall 9 such that the jet 14′ can be directed towards the rim 10 of the conical part 9 by which the complete area under the cover 5 can be flushed.

The parameters for the radii in the corrugated part and the other dimensions as illustrated in FIG. 2d are for illustration only and not limiting for the invention in any way.

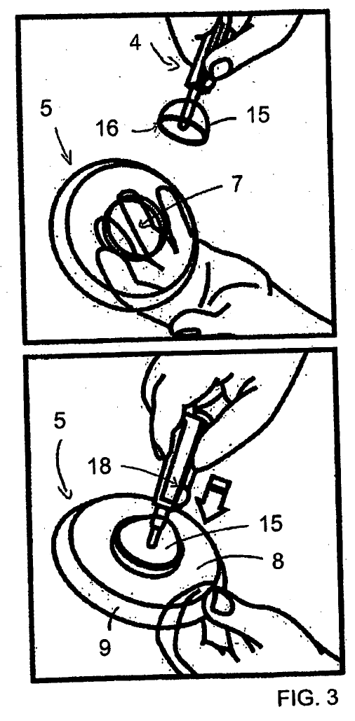

FIG. 3 illustrates the mounting of the cover 5 on the hand piece 4. For simplicity, the cover is shown without corrugation. The hand piece 4 comprises a shield in the form of a solid semi sphere 15 which tightly fits into the central opening 7 of the flange 8 of the cover 5. The edge 16 fits into a recess 17 as shown in the enlarged view in FIG. 2d.

The hand piece 4 may optionally in addition have a regulator 18 for regulation of the amount of flushing fluid and for other parameters, for example pulsation frequency, the size of the jet 14 and flushing pressure.

The solid semi sphere 15 is preferably made of a transparent plastic. The semi sphere 15 may alternatively be substituted by other connectors that fit into a corresponding opening 7. It is preferred that the material is transparent, and optionally, it may include a magnifying lens in order for the medical assistant to better see the surface, for example a wound on a limb of the patient. In certain circumstances, the flexible cover 5, though being fabricated in a transparent material, may not be as clearly transparent as the solid connector/semi sphere 15. Especially in this case, the lens in the solid semi sphere may be useful when the flushing is performed towards the rim 10 of the cover 5 with a tilted hand piece 4 as illustrated in FIG. 4b, because the visual distance through the solid connector 15 to the rim 10 is longer and therefore, details are harder to see for the medical assistant. Thus, a built-in magnifying lens gives a better overview and also makes it easier to see details for the assistant.

A suitable size for the flexible shied has proven to be between 130 mm and 170 mm. The cover may be made of any elastic material, however, polymer, especially silicone, has been observed as useful. The corrugated part has been tested for a polymer a thickness of 0.55 mm, wherein the amplitude for the corrugation was around 3 mm and where the edge region of the flange 8 had a thickness of 1.6 mm. A useful height is between 1 and 5 cm, preferably around 3 cm.

Though the invention has been illustrated for medical usage, especially for cleaning peripheral organs, the cover according to the invention is of general nature and may find application also in other medical and non-medical fields.

The fluid to be provided by the hand held unit 4 would usually be a liquid but the invention is not limited to liquids such that a cover according to the invention also may be used in connection with high pressure gas stream, where the cover holds debris back to prevent spreading of it outside the cover.

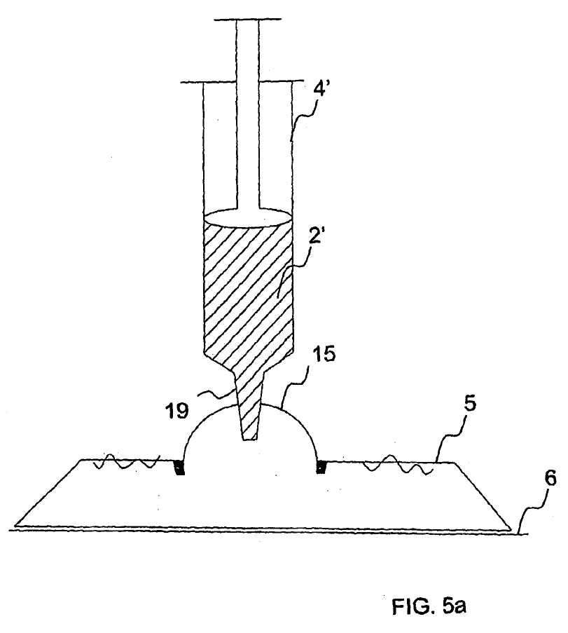

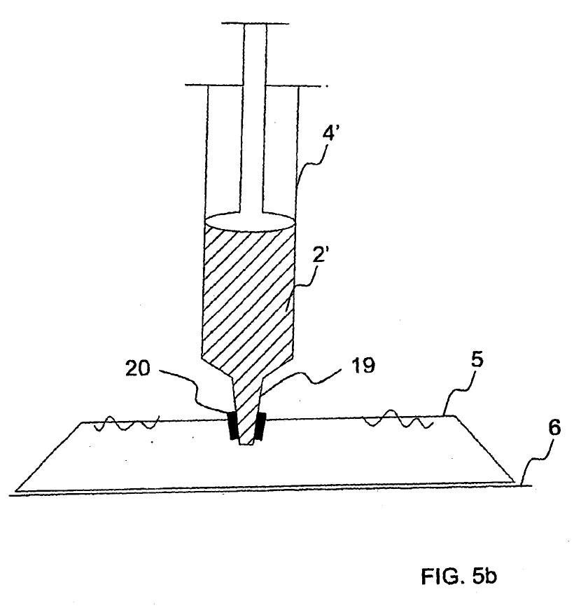

An alternative embodiment of the invention is illustrated in FIGS. 5a and 5b. In this embodiment, the hand-held unit is in the form of a syringe 4′ with a tip 19 in connection with a fluid reservoir 2′ for providing fluid to the surface 6. The syringe 4′ is connected to the cover 5 according to the invention via a connector in the form of a small shield 15 or in the form of a sleeve 20 between the tip 19 and the cover 5. The connector can be of other types as well.

1. An apparatus (1) for flushing a surface (6) with a fluid, the apparatus comprising a fluid supply (2) in connection with a hand held unit (4) for providing a jet (14, 14′) of fluid towards the surface (6), the hand held unit (4) being connected via a connector (15) to a cover (5) with a rim part (9), the rim part being resilient for easy adaptation of the rim (10) to a non-planar surface to be flushed, wherein the hand held unit (4) with the cover (5) has means (11) for changing the direction of the jet (14, 14′) in the cover (5) for flushing an area under the cover which largely corresponds to the entire area covered by the cover.

2. An apparatus according to claim 1, wherein the means for changing the direction of the jet (11) in the cover comprises a flange part (8) of the cover (5) with a flexing zone (11) between the rim part (9) and the connector (15) for large angle tilt of the hand held unit (4) with respect to the rim part (9).

3. An apparatus according to claim 2, wherein the connector (15) comprises a solid, transparent shield.

4. An apparatus according to claim 3, wherein the solid, transparent shield (15) is formed as a semi-sphere.

5. An apparatus according to claim 3, wherein the solid, transparent shield (15) comprises a magnifying lens.

6. A cover (5) for an apparatus according to claim 1, wherein the cover has an opening (7) for receiving a connector (15), a resilient rim part (9) for adapting the rim (10) to a non-planar surface (6) to be flushed, and a flange part (8) with a flexing zone (11) between the rim part (9) and the connector (15) for large angle tilt of the connector (15) with respect to the rim part.

7. A cover according to claim 6, wherein the flexing zone comprises an area with corrugations (11).

8. A cover according to claim 7, wherein the corrugations (11) encircle opening.

9. A cover according to claim 6, wherein at least the rim part (9) of the cover (5) is made of flexible silicone rubber.

10. A cover according to claim 6, wherein the diameter of the rim (10) is between 120 mm and 180 mm, preferably between 130 an 170 mm.
